(19) United States
(12) Patent Application Publication
(10) Pub. No.: US 2009/0175949 A1
(43) Pub. Date: Jul. 9, 2009

(54) MICROPARTICLES FOR DELIVERY TO CELLS AND/OR TISSUES

(75) Inventors: Alexandre Yurievich BOROVKOV, Tempe, AZ (US); Sergei Alex SVAROVSKY, Tempe, AZ (US); Kathryn Frances SYKES, Tempe, AZ (US)

Correspondence Address: Baker Donelson Bearman, Caldwell & Berkowitz, PC, 555 Eleventh Street, NW, Sixth Floor, Washington, DC, 20004, US

(73) Assignee: Arizona Board of Regents Acting for and on Behalf of Arizona State University, Scottsdale, AZ (US)

(21) Appl. No.: 12/339,815

(22) Filed: Dec. 19, 2008

Related U.S. Application Data
(60) Provisional application No. 61/015,584, filed on Dec. 20, 2007.

(51) Int. Cl. A61K 9/14; B32B 15/02

(52) U.S. Cl. 424/490; 428/403

(57) ABSTRACT

Delivery of bioactive molecules (BAMs) to organelles, cells, tissues, or animals with high efficiency is desirable. Novel compositions for highly efficient delivery of bioactive molecules by biolistic methods are described. The novel compositions include novel microparticle cores, microparticle core/BAM complexes, microparticle core/nanoparticle complexes and multilayer-microparticle core/BAM complexes. Any one of these may be further modified to include targeting agents.

This invention was made with government support under 5U54 AI067156-05 awarded by NIH/NIAID. The government has certain rights in the invention.

### INTRODUCTION

The present discovery relates to microparticle cores and microparticle core complexes useful for delivery of bioactive molecules (BAMs) to cells, tissues, organs, organelles or whole organisms such as plants, animals, fungi and yeasts, etc. The present invention allows for significantly higher transfection efficiency and for improved dispersal and reduced aggregation of the microparticles when used for biolistic delivery.

### BACKGROUND TO THE INVENTION

With the exception of freely permeable small molecules, the current list of intracellularly deliverable bioactive molecules is mostly limited to nucleic acids capable of amplifying the delivered signal via replication, transcription and/or translation. Without signal amplification, transfection efficiency is usually too low to induce noticeable changes. Even transcription and/or translation alone, without the replication component, often are not enough to achieve the desired effects, especially on a physiological level.

Biolistic Particle Delivery (Gene Gun) technologies is the preferred method of intracellular delivery. However, current technologies are limited to DNA delivery and rely heavily on particle-mediated delivery protocols where DNA precipitate is trapped on the surface of gold microparticles without chemical attachment. This is achieved by entrapping gold particles into a Ca-DNA/spermidine mesh. The process is difficult to control and is sensitive to numerous environmental factors. Further, the formed mesh is fragile and quickly degrades over time. All this results in inconsistency and low efficiency of intracellular DNA delivery.

Biolistic delivery of DNA by existing conventional methods, which rely on mechanical encrustation of the gold microparticles with a “cake-like” DNA/Calcium/Spermidine precipitate, typically results in very broad DNA per particle distribution and a tendency of the complex to shed off the particles during preparation, storage and their delivery. Attempts to increase DNA load by conventional methods result in greater shedding and aggregation of microparticles. The pattern produced using conventionally fabricated gold microparticles is characterized by large aggregations of microparticles, reducing the number of dispersed microparticles for efficient penetration of cell membranes. Thus, there remains a need for compositions and methods for highly efficient delivery of biologically active molecules (BAMs) to cells that allow for simple preparation, flexibility, consistency, are fully controllable and lead to reproducible BAM loading. The present invention fulfills this need.

### BRIEF DESCRIPTION OF THE DRAWINGS

FIG. 1 shows representative polymers suitable as a second linker molecule in a microparticle core of the present invention: PLL FIG. 1(a), PEI FIG. 1(b) Chitosan FIG. 1(c).

FIG. 2 shows a PEI linker (101) that is covalently attached by an amide bond to the carboxyl moiety of tiopronin (102), the thiol moiety of tiopronin being covalently bound to the gold microparticle (104). Because of the exposed positively charged amines of the PEI polymer, the microparticle core presents a positively charged surface (103).

FIG. 3 shows schematically how the negatively charged backbone (106) of the applied DNA (105) causes it to be linked strongly to the positively charged surface (103) of the microparticle core.

FIG. 4 shows that microparticle cores of the present invention strongly uptake DNA. DNA uptake quickly reaches a plateau of approximately 3 μg DNA per mg Au after 15 minutes.

FIG. 5 compares the dispersal pattern produced upon gene gun delivery into cell culture of gold microparticles encrusted with DNA/Calcium/Spermidine precipitate according to the conventional approach (see FIG. 5(a)) with the pattern produced under equivalent conditions upon delivery of a microparticle core of the present invention with linked DNA (see FIG. 5(b)).

FIG. 6 shows a nanoparticle comprising gold as the nano-carrier and DNA as the BAM, attached to a microparticle core of the present invention.

FIG. 7(a) compares expression levels of a luciferase reporter gene transfected into cells in culture via gene gun delivery of 1 μg plasmid DNA: conventional method (112) (mechanical encrustation of gold microparticles with a DNA/Calcium/Spermidine precipitate); a microparticle core comprising gold and PEI (113); a microparticle core comprising gold and PEI with DNA linked to the PEI (114). FIG. 7(b) is a depiction of an assay used to measure luciferase activity used to test transfection efficiency.

FIG. 8 shows gene gun delivery of a microparticle core/nanoparticle complex (DNA-gold nanoparticle) onto cell culture.

FIG. 9 shows a TEM image of a portion of a microparticle core/nanoparticle complex (DNA-gold nanoparticle) (107) clearly visible on the outline of the microparticle surface.

FIG. 10 represents a multilayer-microparticle core/bioactive molecule complex: a microparticle core with alternating layers of PEI and DNA.

FIG. 11 shows a TEM image of a multilayer-microparticle core/bioactive molecule complex having multiple alternating layers of PEI and DNA. The layers can be seen as a lower density region along the outline of the microparticle core.

FIG. 12(a) depicts a multilayer-microparticle core/bioactive molecule complex with a third linker (118), coupled with agents for membrane targeting, binding, and/or penetration (119). Different biologically active molecules can also be used (120). FIG. 12(b) depicts delivery of the microparticle core carrying with it all or part of the adjacent negatively charged DNA. FIG. 12(c) depicts the accompanying agents for membrane targeting, targeting, and/or penetration (119) mediating localization and/or membrane attachment or penetration by the detaching the DNA-third linker (PEI) polyplex (123).

### SUMMARY OF THE INVENTION

The present invention provides microparticle cores and microparticle complexes suitable for biolistic delivery of a bioactive molecule (BAM) to a cell, organelle, organ, tissue, organism etc. One embodiment of the present invention provides a microparticle core comprising a microparticle, a first linker molecule and a second linker molecule. The first linker molecule comprises a first and second end and the second linker molecule comprises a first and second end. The first end of the first linker molecule is linked to the microparticle and the second end of the first linker molecule is linked to the first end of the second linker molecule. The second end of the second linker molecule is capable of linkage to a first bioactive molecule.

The invention further provides a microparticle core/BAM complex where a BAM is linked to a microparticle of the present invention.

The present invention further provides a microparticle core-nanoparticle complex comprising a microparticle core of the present invention and a nanoparticle. The nanoparticle comprises a nano-carrier molecule linked with a BAM. The nanoparticle is linked to the microparticle core.

The invention further provides a multilayer-microparticle core/BAM complex comprising a microparticle core of the present invention and additional sequential layers of linker molecules and BAMs.

The present invention further provides microparticle core/BAM complexes, microparticle core-nanoparticle complexes and multilayer-microparticle core/BAM complexes of the present invention further comprising a targeting agent, a membrane binding agent or a membrane penetrating agent.

The present invention further provides the use of any of the microparticle cores and complexes of the present invention for delivery to a cell or tissue by biolistic methods.

The present invention also contemplates kits. In one embodiment the kit comprises a microparticle core of the present invention. Kits of the present invention may further comprise one or more BAM, a nano-carrier molecule, a nanoparticle, or additional linkers.

The present invention further provides methods of making microparticle cores and microparticle core complexes of the present invention.

### DETAILED DESCRIPTION OF THE INVENTION

The present invention generally provides novel microparticle cores and microparticle core complexes useful for delivery of bioactive molecules to cells, organelles, tissues, organs or to a whole organism. Bioactive molecules are those molecules that when introduced to a cell, tissue, organ or whole organism influence a physiological change in the cell, tissue, organ or organism. The compositions may be delivered to cells, organelles, tissues, organisms, etc, through various means. However, compositions of the present invention are ideally suited for delivery through biolistic (Gene Gun) delivery. A Gene Gun, or Biolistic Particle Delivery System, is a device for introducing biologically active molecules to a cell or tissue. Biolistic delivery may be in vitro or in vivo and may be to the intracellular or extracellular space, or both. The compositions of the present invention are also suitable for mucosal, intravenous, or other delivery modalities. The use of biolistic technology would be readily understood by one of ordinary skill in the art.

The present invention provides significantly improved efficiency of intracellular transfection/delivery of bioactive molecules (BAMs), significantly improved dispersal of the BAMs within a cell, is applicable for ballistic administration, and is not limited to a specific type of BAM.

### Microparticle Core

The present invention provides a microparticle core comprising a microparticle that is linked to a first linker molecule, a second linker molecule linked to the first linker molecule, and wherein the second linker molecule is capable of linking a BAM. The terms linking or linkage include any attachment means, such as any natural or man-made means of chemically linking, associating or bonding a chemical or biological moiety. Examples, include, but are not limited to covalent or non-covalent bonds, such as ionic or electrostatic association, Van der Waals interaction, hydrophobic bonds, adsorption, antibody-antigen interaction, protein-protein interaction, etc.

The microparticle may be any type of molecule or elemental particle that is non-toxic to the cell and is suitable for the delivery method employed, such as but not limited to biolistic transfection. The microparticle is preferably an inert metal, such as, but not limited to gold, tungsten, platinum, palladium, rhodium, iridium or other precious metals. Most preferably the inert metal comprises gold as it is a particularly suitable choice for biolistic delivery because gold offers the advantage of high density, and is already clinically used.

The microparticle is sized appropriately to allow transfection or delivery to cells, organelles, tissues, etc. and in certain embodiments delivery through biolistic methods. Preferably the microparticle is about 0.25 μm to about 5 μm (or any size or range within). More preferably the microparticle is about 0.5 μm to about 1.5 μm, or 0.75 μm to about 1.5 μm, or 0.9 μm to about 1.3 μm. Most preferably the microparticle is about 1 μm in diameter.

As noted above, the microparticle core further comprises a first linker molecule comprising a first and second end. Preferably the first linker molecule is inert and non-toxic to a cell. The first linker molecule may be any moiety or combinations of moieties having a first end capable of linkage to the microparticle, and the second end capable of linkage to a second linker molecule. The first and second ends may be the same moiety or may be different. The linkage at either end of the first linker molecule may be as described above and in a preferred embodiment, the linkage of the first linker molecule to the microparticle is by covalent or non-covalent means and linkage to the second linker molecule is also by covalent or non-covalent means. In an exemplary embodiment, the first end of the first linker molecule may have a thiol for covalent linkage with the microparticle. The second end of the first linker molecule may be an amine or carboxyl group or other moiety capable of covalent or non-covalent linkage to a second linker molecule. Preferably the first end of the first linker molecule comprises a thiol containing moiety, and the second end of the first linker molecule comprises a carboxyl containing moiety. In a most preferred embodiment, the first linker molecule comprises tiopronin as it is non-toxic to a cell and exposes a thiol linkage for covalent linkage to the microparticle and an amide linkage for covalent linkage to the second linker molecule. Depending on the application, other compositions that are non-toxic to a cell and expose a thiol or other moiety for covalent or non-covalent linkage with the surface of the microparticle and expose an amine, carboxyl or other group suitable for linkage to a chosen second linking molecule may used in lieu of tiopronin.

In certain embodiments, a first linker molecule forms a shell around an inert microparticle, with or without a chemical interaction between the microparticle and the first linker molecule.

The microparticle core further comprises a second linker molecule comprising a first and a second end. The second linker molecule may be any molecule that is non-toxic to a cell, capable of linkage to the first linker molecule, and capable of linkage to a bioactive molecule (BAM) at its second end. The first end of the second linker molecule is preferably capable of covalent or non-covalent linkage to the second end of the first linker molecule. The second end of the second linker molecule is preferably capable of covalent or non-covalent linkage to a bioactive molecule. The first and second ends may be the same moiety or may be different. The second linker molecule may be a hydrophobic, cationic, or anionic molecule.

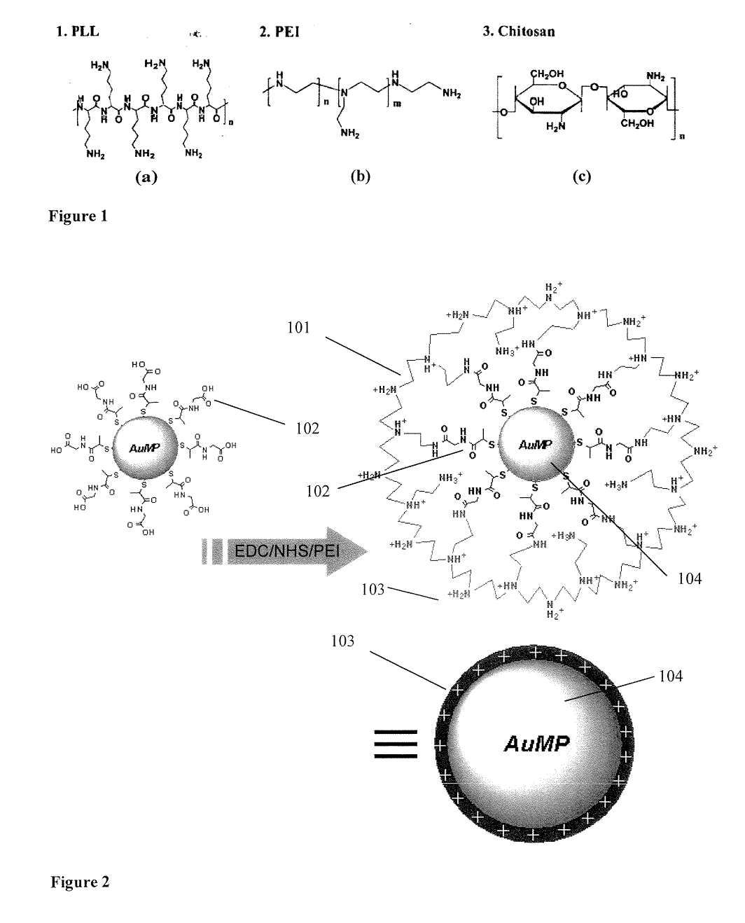

In a preferred embodiment, the second linker molecule may be poly-D,L-lysine (PLL), poly-D,L-arginine (PLA), polyacrylamide, chitosan, polyethyleneimine (PEI) or any other polyelectrolyte or polyampholyte. FIG. 1 depicts the chemical structure of PLL, PEI, and Chitosan. In a most preferred embodiment, the second linker molecule is PEI.

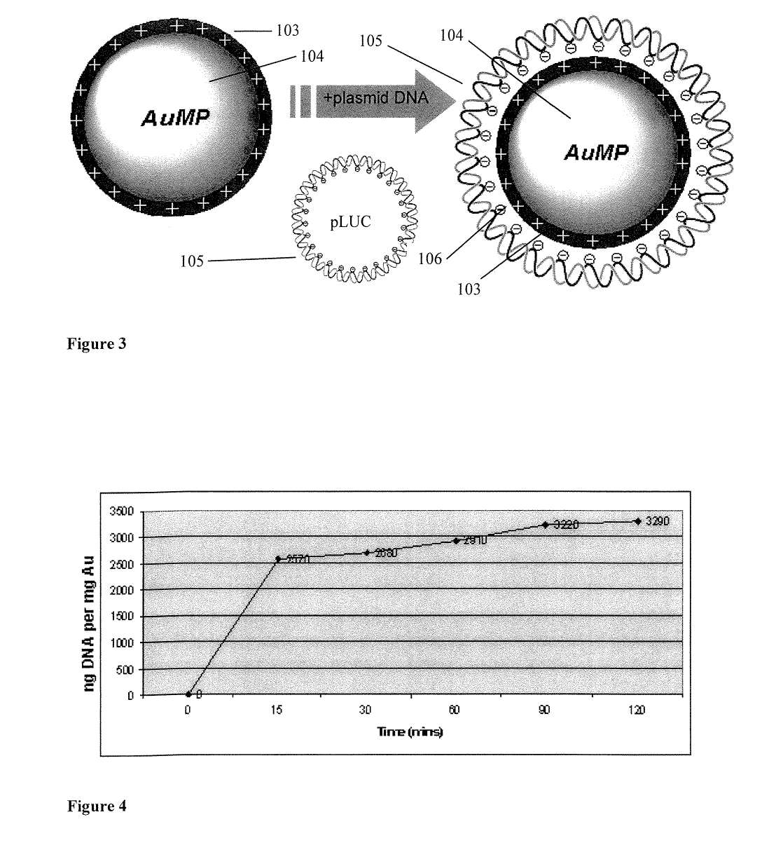

Thus, in one embodiment, the microparticle core contains PEI as the second linker molecule, which is covalently bonded to the first linker, tiopronin, by an amide bond. Because PEI carries a positive charge at its second end, it may be linked to any biologically active molecule (BAM) that is negatively charged through electrostatic attraction. An exemplary embodiment of a microparticle core of the present invention is shown in FIG. 2, which depicts a gold microparticle (104), a first linker comprised of tiopronin (102) and a second linker molecule comprised of PEI (101). The PEI is linked by an amide bond to the carboxyl moiety of tiopronin and the thiol moiety of tiopronin is covalently bonded to the gold microparticle. The PEI linker has a positive charge at its second end due to the exposed amide group that forms the outer-surface of the microparticle core (103). As illustrated schematically in FIG. 3, the negatively charged backbone (106) of the applied DNA (105) causes it to be strongly linked to the positively charged surface (103) of the PEI linker molecule.

Example 1 shows one preparation of an exemplary microparticle core of the present invention. Specifically, Example 1 describes the production of a gold microparticle having tiopronin as a first linker molecule, and PEI as the second linker molecule.

In other embodiments, the second linker molecule may also be negatively charged at its second end for linkage to positively charged BAMs. In another embodiment, the second linker may have any chemical or biological moiety at is second end to allow linkage to a BAM. Further, the BAM can be modified or derivatized to have a chemical or biological moiety that is capable of linking to the second end of the second linker. Obviously, it is desired that the chemical or biological moieties that are used do not affect the ability of the BAM to carry out its desired function as well as not be detrimental to the cell, tissue, etc. For example, the second end of the linker may be derivatized to have a streptavidin moiety that would bind to a biotin moiety on the BAM or vice versa. Other non limiting examples include chemically cleavable linkages, antibody-antigen linkages or protein-protein interactions capable of appropriate modification or derivatization.

The present invention further provides a kit comprising a microparticle, a first linker and a second linker. The microparticle, first and second linkers are as described above.

The present invention also provides a method of making a microparticle core suitable for biolistic delivery of a BAM. The method comprises the steps of mixing a gold microparticle with a solution comprising tiopronin to obtain a tiopronin-gold particle; adding EDC and sulfo-NHS to the tiopronin-gold particle to form an activated tiopronin-gold modified particle; and adding PEI to the activated tiopronin-gold modified particle to form a microparticle core suitable for biolistic delivery of a BAM.

### Microparticle Core-BAM Complex

The present invention further provides a microparticle core-BAM complex, which is a microparticle core as described above, wherein the second end of the second linker molecule is linked to a first bioactive molecule. The bioactive molecule (BAM) may be any molecule such as nucleic acids (i.e. DNA, RNA, siRNA, mRNA, miRNA or antisense nucleotides, proteins, peptides, lipids, polysaccharides, metabolites, drugs, molecular complexes, macromolecular structures or combinations and/or mixtures of any of the foregoing from a naturally occurring or biologically or synthetically designed source. The BAM may be positively or negatively charged, depending on the charge of the second linker molecule used to create the microparticle core, or may be modified to carry a chemical or biological moiety to provide a covalent linkage or a biological association where such linkage or association is feasible.

In a preferred embodiment, charged BAMs are linked the microparticle core by electrostatic attraction. In a most preferred embodiment the BAM is DNA, which is negatively charged, and is linked to a positively charged second end of the second linker molecule of a microparticle core of the present invention by electrostatic attraction. In an exemplary embodiment, the microparticle core is comprised of a gold microparticle, tiopronin as the first linker molecule, and PEI as the second linker molecule. FIG. 3 shows a depiction of a microparticle core with a charged outer layer onto which negatively charged DNA molecules have been linked.

FIG. 4 shows that the charged microparticle cores strongly take up DNA, reaching a plateau of about 3 μg of DNA per mg of microparticle core within about 15 minutes as measured by change in light absorption by DNA in the supernatant (the DNA content of the supernatant is depleted as the DNA adsorbs to the precipitated gold particles). 1 μg of DNA per mg of gold or less is sufficient for adequate intracellular delivery of DNA.

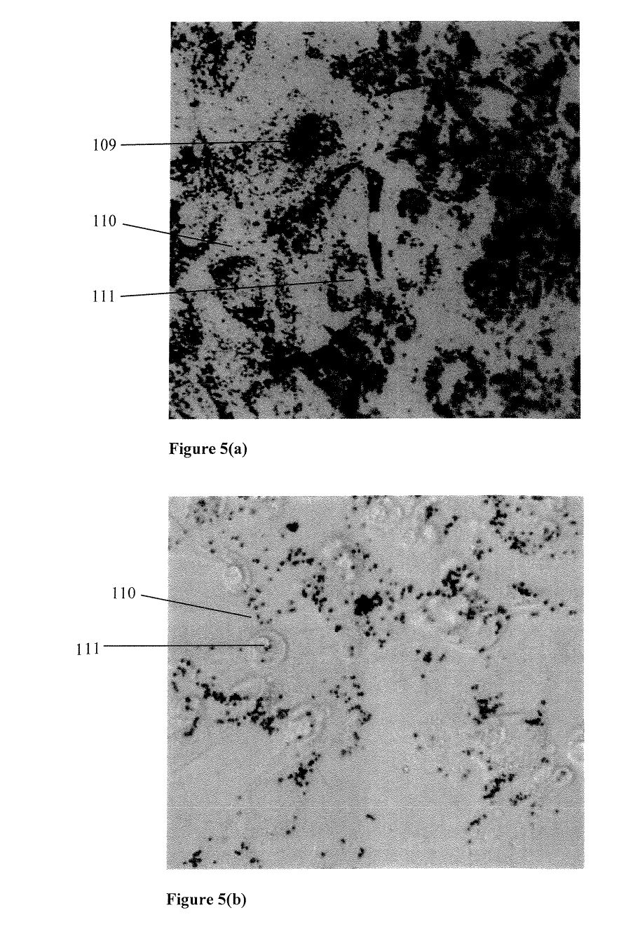

FIG. 5 shows the results of biolistic delivery of either conventionally prepared gold microparticles encrusted with DNA/Calcium/Spermidine (see FIG. 5(a)) or microparticle cores linked with DNA (see FIG. 5(b)) according to the present invention. The pattern produced using the conventionally prepared DNA/Calcium/Spermidine complex is characterized by an uneven dispersal of the microparticles due to large aggregations of the microparticles (109) leaving relatively few dispersed microparticles (110) for penetration of the cells (111). In contrast, the pattern produced by the microparticle core/DNA complexes of the present invention shows better dispersal and reduced aggregation, with most of the particles (110) avoiding aggregation. Because of the better dispersal pattern and less aggregation, biolistic delivery of the particles of the present invention produce higher transfection rates than existing conventional methods.

The present invention further provides a kit comprising a microparticle, a first linker molecule, a second linker molecule and a BAM. The microparticle, first and second linker molecules and BAMs are as described above.

The present invention also provides a method of making a microparticle core-BAM complex as described in Example 1. A microparticle core may be made as described above and in Example 1 and then may then be mixed with a BAM to form a microparticle core/BAM complex in a solution of ethanol. Finally, the ethanol is removed to provide a microparticle core/BAM complex suitable for biolistic delivery. In certain preferred embodiments, the BAM comprises DNA.

The present invention also provides use of the microparticle core-BAM complex in biolistic delivery methods or other suitable transfection methods. Microparticle core-BAM complexes of the present invention may be used in diagnostic, preventative, and therapeutic methods.

### Microparticle Core/Nanoparticle Complex

In another embodiment, the present invention comprises a microparticle core/nanoparticle complex, which is a microparticle core of the present invention linked to at least one nanoparticle. The term “nanoparticle” is not meant herein to limit the minimum or maximum size of the nanoparticle. The term “nano” is used to merely demonstrate that the nanoparticle is smaller than a microparticle core of the present invention.

Nanoparticles of the present invention comprise a nano-carrier molecule that has a linked BAM. The nano-carrier molecule may be any molecule suitable for linking the intended BAM, including, without limitation, metal-based molecules, lipid-based molecules, polymer-based molecules, and biological assemblies (such as, by way of example only, viruses or virus-derived particles). In a preferred embodiment a nano-carrier molecule may be an inert metal such as gold, tungsten, platinum, palladium, rhodium or iridium. In a most preferred embodiment a nano-carrier molecule is cysteamine-modified gold.

After the nano-carrier molecule is linked with a BAM of choice to create a nanoparticle, the resulting nanoparticle is linked to a microparticle core of the present invention to form a microparticle core/nanoparticle complex.

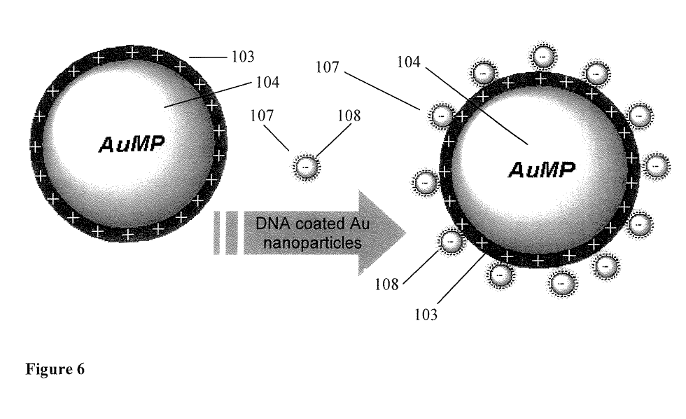

In a preferred embodiment, the BAM is a nucleic acid and in a most preferred embodiment the BAM is DNA and the nano-carrier molecule comprises cysteamine-modified gold. These DNA-nanoparticles are then linked to a microparticle core of the present invention. FIG. 6 shows a schematic depiction of gold nano-carrier molecules onto which DNA has been linked. As illustrated schematically in FIG. 6, the gold nano-carriers (107) are first linked with DNA (108); the DNA linked gold nano-carriers are negatively charged due to the presence of the negatively charged phosphate backbone of the DNA, and bond strongly to the positively charged surface (103) of the PEI-gold-microparticle core (104).

BAM nanoparticles linked to a microparticle core provide greatly increased surface area as compared to a microparticle core/BAM complex alone, and offer the advantage that, upon delivery of the microparticle core to a desired region, the BAM nanoparticle can dissociate from the microparticle core and undergo endocytosis by the target cells or biological tissue, thereby providing effective transfection even without penetration of the target cells by the microparticle core. Further, the nanoparticle provides a greater surface area for delivery of DNA intracellularly.

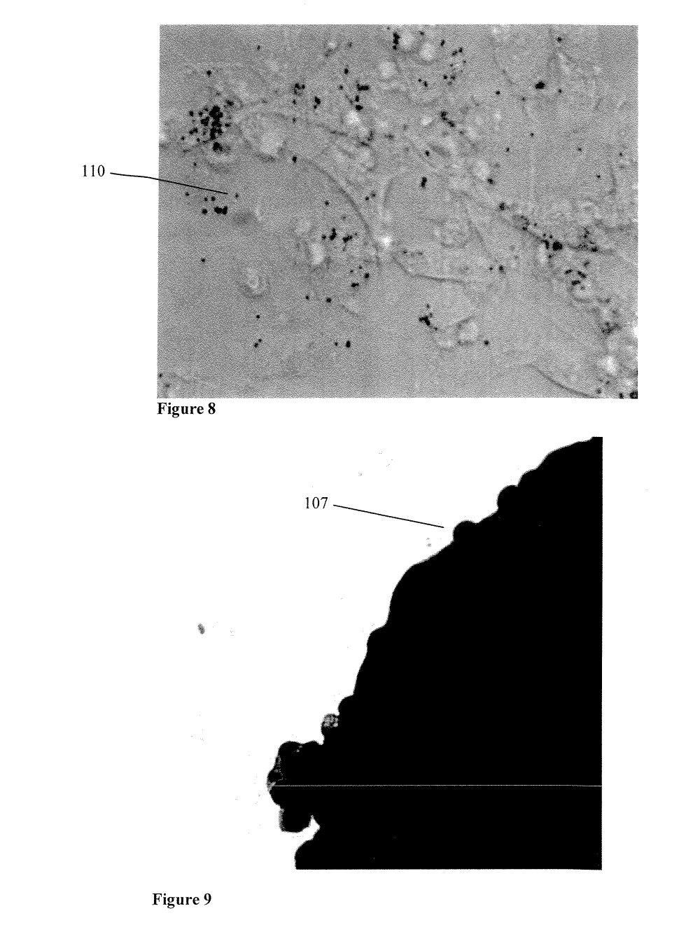

In one example, DNA nanoparticle molecules are electrostatically linked to the surface of an aforementioned microparticle core wherein the second linker is PEI. The microparticle core serves as a carrier for the smaller DNA nanoparticles. Specifically, Example 2 describes the production of a cysteamine modified gold nano-carrier to which DNA is linked (thus providing a DNA-nanoparticle). The DNA nanoparticles are then linked to a microparticle core of the present invention. These structures may then be delivered to cells or tissues by any means, preferably biolistic means. As shown in FIG. 8, Gene Gun delivery of a microparticle core/nanoparticle complex of the present invention (a microparticle core comprising PEI as the second linker molecule and a nanoparticles comprised of DNA and cysteamine-modified gold nano-carriers) onto cell cultures produced excellent dispersal of particles, with very little aggregation. Nearly all particles distributed over the target area. FIG. 9, a transmission electron microscopy (TEM) image, shows that the microparticle core is far from saturated with nanoparticles (107), indicating that the quantity of DNA delivered per microparticle core could be substantially increased if needed.

The present invention further provides a kit comprising a microparticle, a first linker molecule, a second linker molecule, and a BAM nanoparticle, all as described above.

The present invention also provides a method of making a microparticle core/nanoparticle complex suitable for biolistic delivery of a BAM, as described in Example 2. The method comprises a method of making a microparticle core/nanoparticle complex suitable for biolistic delivery of a BAM, comprising the steps of: a) mixing a gold microparticle with a solution comprising tiopronin to obtain a tiopronin-gold particle; b) adding EDC and sulfo-NHS to the tiopronin-gold particle to form a tiopronin-gold modified particle; c) adding PEI to the tiopronin-gold modified particle to form a microparticle core; d) forming a cysteamine-modified gold nanocarrier molecule comprising adding cysteamine in water to a solution of HAuCl₄ to form a cysteamine-HAuCl₄ mixture, and then adding NaBH₄ to the cysteamine-HAuCl₄ mixture to form the cysteamine-modified gold nanocarrier molecule; e) adding the cysteamine-modified gold nanocarrier molecule from step d to a BAM to form a nanoparticle; and f) adding the nanoparticle from step e to the microparticle core from step c to form a microparticle core/nanoparticle complex suitable for biolistic delivery of the BAM.

In certain preferred embodiments, the BAM comprises DNA.

The present invention also provides the use of the microparticle core-BAM complex in biolistic delivery methods or other suitable transfection methods. Microparticle core-BAM complexes of the present invention may be used in diagnostic, preventative, and therapeutic methods.

### Multilayer-Microparticle Core/BAM Complex

In another embodiment, the present invention provides a multilayer-microparticle core/BAM complex that is comprised of a microparticle core, a first BAM, a third linker molecule (the first and second linker molecule being part of the microparticle core) and a second BAM. The third linker molecule has a first end linked to the first BAM and a second end linked to a second BAM. The multilayer-microparticle core/BAM complex can have additional layers of linker molecules and BAMs. For example, after the second BAM, a fourth linker may be used to link to a third BAM and this sequential layering may continue such that a plurality of linkers and BAMs may be sequentially layered on a microparticle core. The microparticle core, the linker molecules and the BAMs, as well as the linkage are as described above. Example 3 shows the preparation of a multilayer-microparticle core/BAM complex of the present invention.

The successive layers of linkers and the successive layers of BAMs may be the same compositions, or different combinations of linkers and BAMs applied in different layers. Appropriate selection of these combinations of linkers and BAMs will allow precise regulation of the rate at which BAMs are released in the target cell or tissue, in turn enabling precisely timed intracellular or extracellular release of a substance or series of BAMs in succession.

In a preferred embodiment the alternating layer-by-layer complex is composed of positively charged microparticle cores and negatively charged BAMs. In a most preferred embodiment the layer-by-layer complex comprises PEI linker molecules and DNA BAMs. The layered complex not only allows higher loading capacities but also provides the flexibility of using different DNAs slowly released over pre-programmed time lapses.

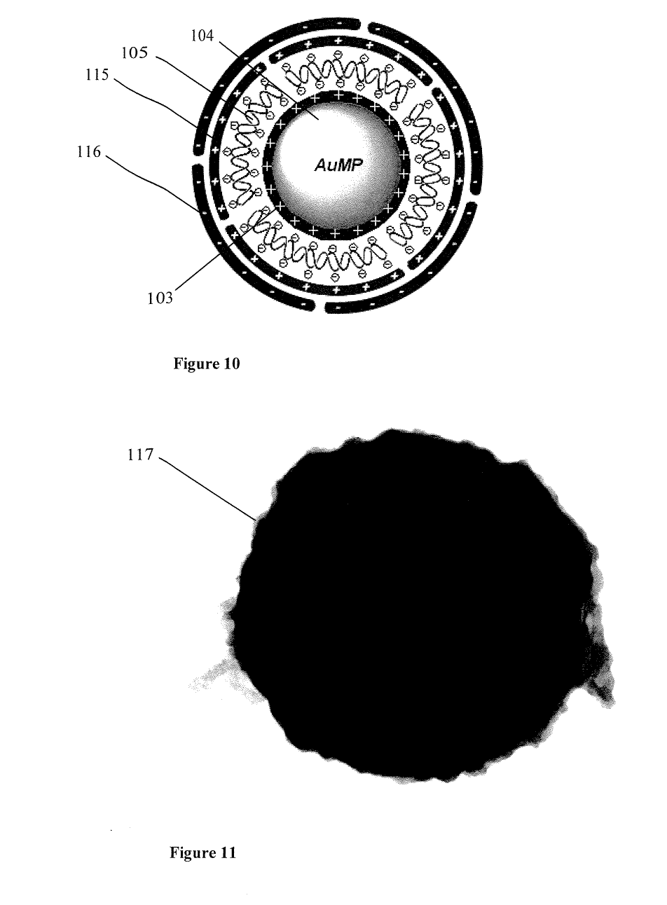

FIG. 10 is a depiction of a multilayer-microparticle core/BAM complex of the present invention. In this depiction, microparticle cores are comprised of PEI as the second linker and have a linked DNA. A second layer of PEI is applied as a third linker and another layer of DNA is applied. This process may be repeated to obtain additional alternating layers of PEI and DNA. The DNA may be replaced with other anionic polyelectrolytes including non limiting examples of hyaluronate, alginate, and heparan sulfate. As illustrated schematically in FIG. 10, gold-tiopronin-PEI microparticles cores are coated with alternating layers of PEI and DNA, enabling timed sequential delivery of each DNA layer as the PEI layers are gradually degraded and/or detached. A PEI layer (103) is applied to the gold-tiopronin-PEI microparticle (104) and DNA (105) is applied. A second layer of PEI (115) is applied, producing a new positively charged outer layer, to which a second layer of DNA (116) is applied.

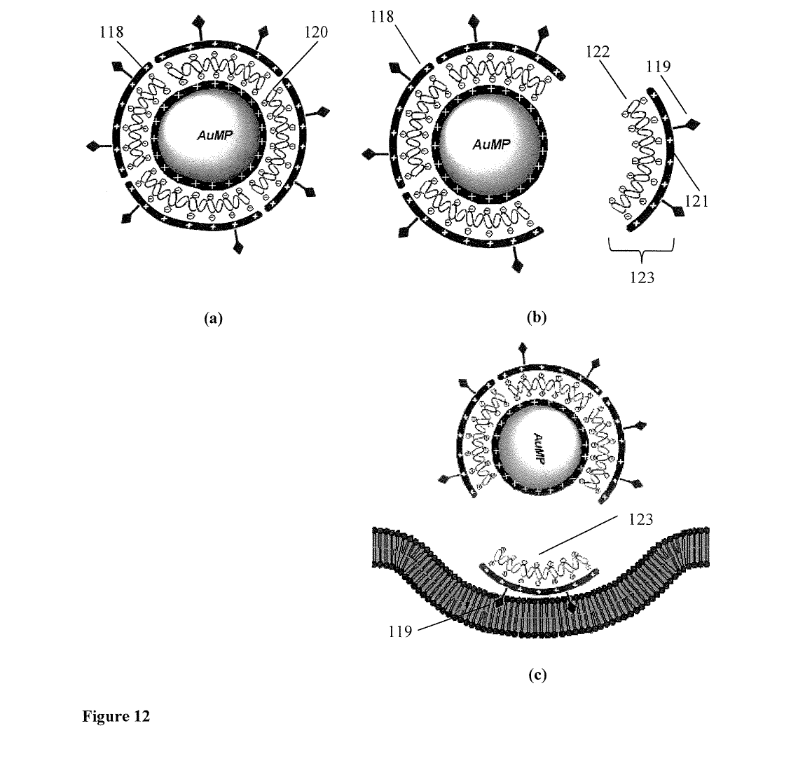

FIG. 11 is a transmission electron microscopy (TEM) picture of a gold/tiopronin/PEI microparticle core with alternating layers or PEI and DNA prepared as set forth in Example 3. The applied material can be seen as a lower density region along the outline of the gold microparticle.

In another embodiment, the present invention provides a kit comprising a microparticle, a first linker molecule, a second linker molecule, and at least one additional linker molecule. The microparticle and linker molecules are as described above.

The present invention also provides a method of making a multilayer-microparticle core/BAM complex as described in Example 3. The method comprises the steps of: a) mixing a gold microparticle with a solution comprising tiopronin to obtain a tiopronin-gold particle; b) adding EDC and sulfo-NHS to the tiopronin-gold particle to form a tiopronin-gold modified particle; c) adding PEI to the tiopronin-gold modified particle to form a microparticle core to form a microparticle core; d) adding a first BAM to the microparticle core to form a microparticle core/BAM complex; e) adding PEI to the complex of step d to form a microparticle core/BAM complex/PEI complex; and f) adding a second BAM to the a microparticle core/BAM complex/PEI complex of step e to form a multilayer-microparticle core/BAM complex suitable for biolistic delivery of a BAM.

The present invention also provides the use of the multilayer-microparticle core/BAM complex in biolistic delivery methods or other suitable transfection methods. Multilayer-microparticle core/BAM complexes of the present invention may be useful in diagnostic, preventative, and therapeutic methods.

Microparticle Cores and/or Microparticle Complexes Having Targeting Agents

The present invention further contemplates the use of agents for site specific targeting such as agents for membrane targeting, membrane binding, and/or membrane penetration. Such agents include, but are not limited to, nucleic acids, oligonucleotides, polynucleotides, polysaccharides, oligosaccharides, receptors, ligands, portions of receptors or ligands, antibodies or portions thereof, enzymes or portions thereof, proteins and peptides, liposomes, chemicals or drugs, or other compositions that direct targeting of BAMs to intended locations. These agents for membrane targeting, binding, and/or penetration mediate localization to specific cells, organelles, tissues or other specified locations. By using such agents the microparticle cores or microparticle core complexes of the present invention can be useful for site specific delivery for therapeutic or diagnostic applications.

The targeting agent may be attached to any portion of the microparticle core, the nanoparticle, the BAM, or the linker molecule. Preferably the targeting agent is attached to the outermost layer of a microparticle or microparticle complex of the present invention so the targeting agent is accessible and exposed to the area to be targeted. Attachment of the targeting agent is as described above regarding linking of a linker molecule to a BAM, i.e. covalent, ionic, hydrophobic, molecular binding/affinity, etc.

The use of a targeting agent is illustrated schematically in FIG. 12, where a positively charged third linker (118) is coupled with agents for membrane targeting, binding, and/or penetration (119). After delivery of microparticle, regions of the outer positively charged third linker surface (121) detach from the microparticle core carrying with them all or part of the adjacent negatively charged DNA (122) (see FIG. 12(b)). The targeting agents (119) mediate localization of and/or membrane penetration by the detaching DNA-third linker (PEI) polyplex (123) (see FIG. 12(c)).

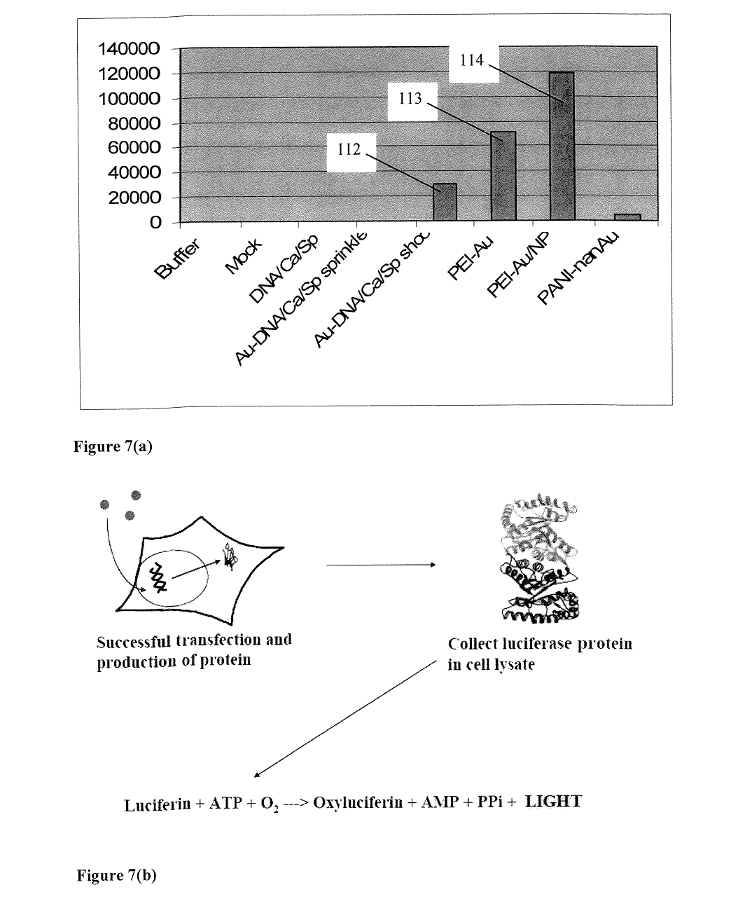

All of the microparticle cores or microparticle core complexes of the present invention preferably provide the advantages of: 1) simple preparation of the components; 2) simplicity, robustness and reproducibility of the manufacturing process; 3) ability to bind orderly, independently or in parallel BAMs of different nature, conformation and size; 4) flexibility of formulation; 5) control of the rate of order of BAM release; 6) low BAM losses during preparation, storage, and use of the complexes; 7) fully controllable, consistent and reproducible BAM loading; and 8) conferring efficient BAM delivery. These advantages may open new opportunities for high-throughput gene delivery technologies. FIG. 7 provides the results of an assay where conventional biolistic particles are compared with a microparticle core and microparticle complexes of the present invention. The results in FIG. 7 are from an assay that measures expression levels of a luciferase reporter gene transfected into cells in culture via gene gun delivery of 1 μg plasmid DNA. The assay measures light emission by luciferin in the presence of luciferase in cell lysate, ATP, and oxygen. The conventional biolistic particles (112) (mechanical encrustation of gold microparticles with a DNA/Calcium/Spermidine precipitate) are compared with a microparticle cores having PEI as the second linker (Example 1) (113), and a microparticle core/nanoparticle complex [(microparticle core having PEI as the second linker linked) linked to DNA nanoparticles (gold nanocarrier molecule with DNA) (Example 2) (114). As FIG. 7 shows, the luciferase expression level upon transfection by gene gun delivery of the microparticle core/DNA complex or of the microparticle core/DNA nanoparticle complex was more than two-fold higher than for the existing conventional method. As FIG. 7 indicates, no detectable levels of luciferase were observed for the four negative controls: buffer alone; mock (gene gun delivery of PEI-coated particles alone, without DNA); application of DNA/Calcium/Spermidine precipitate without biolistic delivery; and non-biolistic delivery of gold particles encrusted with DNA/Calcium/Spermidine precipitate by sprinkling the particles on the culture.

### EXAMPLES

### Example 1

### Preparation of a Gold Microparticle Core and Gold Microparticle Core/BAM Complex

In this example, a gold microparticle core was prepared, after which DNA was applied, to form a microparticle core/BAM complex.

Preparation of a Microparticle Cores with Tiopronin as a First Linker Molecule and PEI as a Second Linker Molecule (“PEI-Tiopronin-AuMP”)

Clean 50 mg of gold microparticles (AuMP, diameter=1 μm) with piranha solution (450 μl H₂SO₄+150 μl H₂O₂) for 1 hour and wash 3 times with double distilled water. Add 0.5 ml of 50 mg/ml water solution of tiopronin (N-(2-mercaptopropionyl) glycine, (CAS#1953-02-2) to the gold microparticles and stir at room temperature for 1 hour. Pellet gold particles by brief centrifugation, aspirate the supernatant, and wash the pellet once with 0.1M MES, pH6.0, 0.5M NaCl. Add 10 mg EDC and 27 mg sulfo-NHS (Pierce, Inc) to the tiopronin-modified particles and incubate at room temperature with vigorous stirring for 15 minutes. Briefly spin gold particles, aspirate the supernatant, and resuspend the pellet in 1 ml of PBS supplemented 50% weight/volume PEI (MW750,000, Sigma#P3143, CAS#9002-98-6). Vigorously stir the suspension for 1 hour at room temperature.

Application of DNA to produce PEI-Tiopronin-AuMP-DNA

Add 3 μg DNA in water to 3 mg MES/NaCl washed PEI-AuMP, bring total volume to 1 ml with 0.1M MES, pH 6.0, 0.5M NaCl solution and incubate at 0° C. for one hour. Briefly spin gold particles by centrifugation, aspirate the supernatant, and wash gold with several changes of absolute ethanol. The resulting PEI-AuMP-DNA complex can be used immediately or stored under ethanol.

### Bullet Preparation for Biolistic Delivery

Resuspend the ethanol washed complex from above in a desirable volume of absolute ethanol, optionally supplemented with PVP (0.1-0.05 g/ml). For DNA containing complexes, the golden slurry is adjusted to equivalent of 1 μg DNA per 55 μl of the suspension, vortex the suspension to ensure even gold distribution. Immediately transfer the suspension into a piece of Teflon tubing and let the gold particles settle on the side of the tubing. Gently drain the ethanol and dry the gold by gently blowing dry inert gas through the tubing.

### Example 2

### Preparation of Microparticle Core/Nanoparticle Complex

In this example, gold containing microparticles cores were prepared according to Example 1. Cysteamine-modified gold nanoparticles (cys-AuNP) are prepared and linked to DNA, and these are linked to the gold containing microparticle cores, to form a microparticle core/nanoparticle complex.

### Preparation of PEI-Tiopronin Modified Gold Microparticles (PEI-Tiopronin-AuMP)

See protocol under Example 1 above.

### Preparation of Cysteamine-Modified Gold Nanoparticles (Cys-AuNP)

Add 0.4 ml of 213 nM cysteamine in water to 40 ml 1.42 mM HAuCl₄ in water and stir vigorously at room temperature for 10 minutes. The mixture should turn deep yellow upon the addition of cysteamine. Quickly add 10 μl of 10 mM NaBH₄ in water to the mix and continue vigorous stirring for an additional 15 min (mixture should immediately turn brown upon addition of the borate). The suspension can be used immediately or stored for up to 2 months at 5° C.

### Application of Cys-AuNP to PEI-AuMP to Produce PEI-AuMP-(cys-AuNP-DNA)

Add 3 μg DNA in 10 μl H₂O to 170 μl of cys-AuNP solution (0.3 μg/μl, 170×0.3=51 μg in gold) to create Au:DNA ratio 17:1, incubate the wine-red mixture for 5 minutes at 0° C. Add 3 mg of PEI-AuMP (0.1 mg/μl) in 30 μl 0.1M MES, pH6.0, 0.5M NaCl to the formed cys-AuNP-DNA complex and incubate the mixture for 1 hour at 0° C. Adsorption of the wine-red colored cys-AuNP-DNA complexes should discolor the supernatant and change color of PEI-AuMP to pink. Briefly spin gold particles by centrifugation, aspirate the supernatant, and wash gold with several changes of absolute ethanol. For best results PEI-AuMP-(Cys-AuNP-DNA) complex should be used immediately.

### Bullet Preparation for Biolistic Delivery

See protocol under Example 1 above.

### Example 3

### Preparation of a Multilayer-Microparticle Core/BAM Complex

In this example, gold microparticles were prepared according to Example 1, DNA was applied, and additional alternating layers of PEI and DNA were applied to produce a multilayer-microparticle core/BAM complex.

### Preparation of Gold Microparticles for PEI Application

Clean 50 mg of gold microparticles (AuMP, diameter=1 μm) with piranha solution (450 μl H₂SO₄ plus 150 μl H₂O₂) for 1 hour and wash 3 times with double distilled H₂O. Add 0.5 ml of 50 mg/ml water solution of tiopronin (N-(2-mercaptopropionyl) glycine (CAS# 1953-02-2) to the gold and stir at room temperature for 1 hour. Pellet the gold particles by brief centrifugation, aspirate the supernatant, and wash the pellet once with 0.1M MES, pH6.0, 0.5M NaCl. Add 10 mg EDC and 27 mg sulfo-NHS (Pierce, Inc) to the tiopronin-modified particles and incubate at room temperature with vigorous stirring for 15 minutes.

Application of Alternating layers of PEI and DNA

The following steps were repeated seven times. Briefly spin the gold particles, aspirate the supernatant, and resuspend the pellet in 1 ml of PBS supplemented 50% w/v PEI (MW 750,000, Sigma#P3143, CAS#9002-98-6). Vigorously stir the suspension for 1 hr at RT. Add 3 μg DNA in water to 3 mg MES/NaCl washed product of the preceding step, bring the total volume to 1 ml with 0.1M MES, pH6.0, 0.5 M NaCl solution, and incubate at 0° C. for 1 hour. Briefly spin the gold particles by centrifugation, aspirate the supernatant, and wash the gold with several changes of absolute ethanol.

### Bullet Preparation for Biolistic Delivery

See protocol under Example 1 above.

1. A microparticle core comprising a microparticle, a first linker molecule and a second linker molecule;
wherein the first linker molecule comprises a first and second end;
wherein the second linker molecule comprises a first and second end;
wherein the first end of the first linker molecule is covalently linked to the microparticle; and
wherein the second end of the first linker molecule is covalently linked to the first end of the second linker molecule; and
wherein the second end of the second linker molecule is capable of linkage to a first bioactive molecule.

2. (canceled)

3. The microparticle core of claim 2 wherein the first linker molecule forms a shell around the microparticle.

4. The microparticle core of claim 1 wherein the microparticle comprises an inert metal.

5. (canceled)

6. The microparticle core of claim 1 wherein the microparticle is about 1 μm in diameter.

7-15. (canceled)

16. The microparticle core of claim 1 wherein the microparticle comprises gold, wherein the first linker comprises tiopronin and wherein the second linker comprises PEI.

17. A microparticle core-bioactive molecule (BAM) complex comprising the microparticle core of claim 1 further comprising a first bioactive molecule, wherein the first bioactive molecule is linked to the second end of the second linker molecule.

18-22. (canceled)

23. A microparticle core-nanoparticle complex comprising the microparticle core of claim 1 further comprising a nanoparticle comprising a nano-carrier molecule linked with a BAM, wherein the nanoparticle is linked to the microparticle core.

24-27. (canceled)

28. The microparticle core-nanoparticle complex of claim 23 wherein the nano-carrier molecule comprises cysteamine-modified gold and the BAM comprises DNA.

29. The microparticle core-nanoparticle complex of claim 28, wherein the microparticle comprises gold, wherein the first linker comprises tiopronin and wherein the second linker comprises PEI.

30. A multilayer-microparticle core/BAM complex comprising
a) the microparticle core of claim 1; and
b) a third linker molecule having a first and second end and a second bioactive molecule, wherein the first end of the third linker molecule is linked to the first BAM; and wherein the second end of the third linker molecule is linked to a second BAM.

31. The multilayer-microparticle core/BAM complex of claim 30 further at least a fourth linker molecule and at least a third BAM.

32. The multilayer-microparticle core/BAM complex of claim 30 further comprising an additional sequential layer of a linker molecule and a BAM.

33-36. (canceled)

37. The microparticle core/BAM complex of claim 17 further comprising a targeting agent, a membrane binding agent or a membrane penetrating agent.

38. The microparticle core-nanoparticle complex claim 23 further comprising a targeting agent, a membrane binding agent or a membrane penetrating agent.

39. The multilayer-microparticle core/BAM complex of claim 30 further comprising a targeting agent, a membrane binding agent or a membrane penetrating agent.

40-45. (canceled)

46. A method of making a microparticle core of claim 1 suitable for biolistic delivery of a BAM, comprising the steps of:
a) mixing a gold microparticle with a solution comprising tiopronin to obtain a tiopronin-gold particle;
b) adding EDC and sulfo-NHS to the tiopronin-gold particle to form a tiopronin-gold modified particle; and
c) adding PEI to the tiopronin-gold modified particle to form a microparticle core suitable for biolistic delivery of a BAM.

47-48. (canceled)

49. A method of making a microparticle core/nanoparticle complex suitable for biolistic delivery of a BAM, comprising the steps of:
a) mixing a gold microparticle with a solution comprising tiopronin to obtain a tiopronin-gold particle;
b) adding EDC and sulfo-NHS to the tiopronin-gold particle to form a tiopronin-gold modified particle;
c) adding PEI to the tiopronin-gold modified particle to form a microparticle core;
d) forming a cysteamine-modified gold nanocarrier molecule comprising adding cysteamine in water to a solution of HAuCl₄ to form a cysteamine-HAuC₁₄ mixture, and then adding NaBH₄ to the cysteamine-HAuCl₄ mixture to form the cysteamine-modified gold nanocarrier molecule;
e) adding the cysteamine-modified gold nanocarrier molecule from step d to a BAM to form a nanoparticle; and
f) adding the nanoparticle from step e to the microparticle core from step c to form a microparticle core/nanoparticle complex suitable for biolistic delivery of the BAM.

50. (canceled)

51. A method of making a multilayer-microparticle core/BAM complex suitable for biolistic delivery of a BAM, comprising the steps of:
a) mixing a gold microparticle with a solution comprising tiopronin to obtain a tiopronin-gold particle;
b) adding EDC and sulfo-NHS to the tiopronin-gold particle to form a tiopronin-gold modified particle;
c) adding PEI to the tiopronin-gold modified particle to form a microparticle core;
d) adding a first BAM to the microparticle core to form a microparticle core/BAM complex;
e) adding PEI to the complex of step d to form a microparticle core/BAM complex/PEI complex; and
f) adding a second BAM to the a microparticle core/BAM complex/PEI complex of step e to form a multilayer-microparticle core/BAM complex suitable for biolistic delivery of a BAM.

52-53. (canceled)
